(19) United States
(12) Patent Application Publication
(10) Pub. No.: US 2018/0218794 A1
(43) Pub. Date: Aug. 2, 2018

(54) QUANTIFICATION OF BLOOD LOSS ON THE BASIS OF COMPUTED TOMOGRAPHY WITH A DIRECTLY CONVERTING DETECTOR

(71) Applicant: Siemens Healthcare GmbH, Erlangen (DE)

(72) Inventors: Philipp HOELZER, Bubenreuth (DE); Steffen KAPPLER, Effeltrich (DE); Sebastian SCHMIDT, Weisendorf (DE)

(73) Assignee: Siemens Healthcare GmbH, Erlangen (DE)

(21) Appl. No.: 15/874,007

(22) Filed: Jan. 18, 2018

(30) Foreign Application Priority Data
Jan. 31, 2017 (DE) 102017201543.8

(51) Int. Cl. G16H 80/00; G16H 30/40; G16H 50/20; G06T 7/00; A61B 6/03; A61B 6/00

(52) U.S. Cl. CPC G16H 80/00; G16H 30/40; G16H 50/20; G06N 99/005; A61B 6/032; A61B 6/507; A61B 6/5217; G06T 7/0012

(57) ABSTRACT

A system and a method are for quantifying blood loss on the basis of contrast-agent based computed tomography imaging of the torso of a patient. To this end, image data of a computed tomograph is firstly read in, in order thereupon to apply an image analysis method for automatically detecting accumulations of blood. A differentiation method is then carried out to differentiate between pathological and physiological accumulations of blood and a quantification algorithm for calculating and outputting a blood loss value for the pathological accumulations of blood.

### PRIORITY STATEMENT

The present application hereby claims priority under 35 U.S.C. § 119 to German patent application number DE 102017201543.8 filed Jan. 31, 2017, the entire contents of which are hereby incorporated herein by reference.

### FIELD

At least one embodiment of the present invention focuses on the fields of medical image processing and computed tomography technology and concerns the determination of blood losses in the case of a polytrauma.

### BACKGROUND

In medicine, the term polytrauma means when a patient is suffering a number of life-threatening injuries at the same time; this is very typical after a road traffic accident for instance. Typically such patients are primarily examined using CT (computed tomography). One frequent problem with these patients involves multiple internal bleeding. While with external bleeding, the blood loss quantity is directly visible and can also be stopped from the outside (e.g. by means of compression), internal bleeding sometimes requires an operation, and the extent of the blood loss can only be assessed very poorly in the short term. Internal bleeding can be identified using computed tomography. For this purpose contrast agent is normally administered. The leakage of the contrast agent from the vessels is then visible in the image and allows for a high quality assessment of the bleeding. A quantitative evaluation of the bleeding would however be necessary in order to decide on the treatment; particularly if there are several bleeding sites, which can only be dealt with one by one operatively and treating the bleeding has to follow an order of priority. It is also very relevant to know the speed at which the patient is losing blood, in order to be able to start a treatment with volume replacement or whole blood. At the same time, this information must however be available within a few seconds in order to still be relevant.

It is known within the prior art to measure the hemoglobin content (Hb) in the blood using laboratory tests. In the long term this value is a good quantitative measure of the blood loss, but hardly reduces acutely because the blood volume reduces acutely but the composition remains unchanged. Only when the volume loss is replaced does the Hb content reduce. Similarly the blood pressure is not a suitable measure of an acute blood loss, since it namely reduces on account of the volume loss (shock), but due to the counter regulation of the heart circulation system, this is not a suitable measure of the blood loss.

Furthermore, it is known that the volumes of hematomas can be determined in the image data by segmentation—the manual identification and segmentation of these hematomas is however completely inconceivable in the short time available.

### SUMMARY

At least one embodiment of the present invention provides a method and/or a system which improves the known method for quantitative determination of the blood loss and in particular allows for a concrete, number-based determination of the blood loss per individual lesion in the shortest time possible.

In at least one embodiment, a method and/or system are disclosed for quantifying blood loss on the basis of contrast agent-based computed tomography imaging of the torso of a patient. Further embodiments of the method with further features can be found in the dependent claims.

According to one embodiment, a method for quantifying blood loss on the basis of contrast agent-based computed tomography imaging of the torso of a patient, comprises:
Reading-in the image data of the imaging;
Applying an image analysis method for automatically detecting pathological and physiological accumulations of blood;
Carrying out a method of differentiation for differentiating between the detected pathological and physiological accumulations of blood and for determining the pathological accumulations of blood; and
Carrying out a quantification algorithm for calculating and outputting a blood loss value for the accumulations of blood determined as pathological.

According to a further embodiment, a quantification system is disclosed for quantifying blood loss on the basis of image data of a computed tomograph of contrast-agent-based computed tomography imaging of the torso of a patient. The quantification system comprises:
An image data interface for reading in the image data of the computed tomograph;
An analyser for automatically detecting intravascular (physiological) and extravascular (pathological) accumulations of blood;
A differentiator, which is determined to differentiate between pathological and physiological accumulations of blood.
A quantifier, which is determined for calculating and outputting a blood loss value for the pathological accumulations of blood.

### BRIEF DESCRIPTION OF THE DRAWINGS

Example embodiments of the invention with further features and advantages are displayed in the drawings and are described in more detail below.

In the drawings:

FIG. 1 shows a schematic view of a computed tomography image with extravascular and intravascular bleed sites;

FIG. 2 shows a flow chart of a method according to a preferred embodiment of the invention;

FIG. 3 shows an example display of result data relating to the blood loss values in annotated form; and

FIG. 4 shows a block diagram of a quantification system.

### DETAILED DESCRIPTION OF THE EXAMPLE EMBODIMENTS

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

Various example embodiments will now be described more fully with reference to the accompanying drawings in which only some example embodiments are shown. Specific structural and functional details disclosed herein are merely representative for purposes of describing example embodiments. Example embodiments, however, may be embodied in various different forms, and should not be construed as being limited to only the illustrated embodiments. Rather, the illustrated embodiments are provided as examples so that this disclosure will be thorough and complete, and will fully convey the concepts of this disclosure to those skilled in the art. Accordingly, known processes, elements, and techniques, may not be described with respect to some example embodiments. Unless otherwise noted, like reference characters denote like elements throughout the attached drawings and written description, and thus descriptions will not be repeated. The present invention, however, may be embodied in many alternate forms and should not be construed as limited to only the example embodiments set forth herein.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “exemplary” is intended to refer to an example or illustration.

When an element is referred to as being “on,” “connected to,” “coupled to,” or “adjacent to,” another element, the element may be directly on, connected to, coupled to, or adjacent to, the other element, or one or more other intervening elements may be present. In contrast, when an element is referred to as being “directly on,” “directly connected to,” “directly coupled to,” or “immediately adjacent to,” another element there are no intervening elements present.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Before discussing example embodiments in more detail, it is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments of the present invention. This invention may, however, be embodied in many alternate forms and should not be construed as limited to only the embodiments set forth herein.

Units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one embodiment of the invention relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

According to one embodiment, a method for quantifying blood loss on the basis of contrast agent-based computed tomography imaging of the torso of a patient, comprises:
Reading-in the image data of the imaging;
Applying an image analysis method for automatically detecting pathological and physiological accumulations of blood;
Carrying out a method of differentiation for differentiating between the detected pathological and physiological accumulations of blood and for determining the pathological accumulations of blood; and
Carrying out a quantification algorithm for calculating and outputting a blood loss value for the accumulations of blood determined as pathological.

In one advantageous embodiment of the invention, the computed tomography imaging is carried out using a directly converting detector, which can comprise a semiconductor layer, for instance, which can directly distinguish the different energy levels of photons. X-ray detectors with a directly converting layer (e.g. made from semiconductor material) allow for a quantitative and energy-selective acquisition of individual x-ray quanta. With this type of x-ray detectors, an incident x-ray quanta in the semiconductor layer generates free charge carriers in the form of electron-hole pairs on account of partly multi-level physical interaction processes with a semiconductor material. Semiconductor materials in the form of CdTe, CdZnTe, CdTeSe, CdZnTeSe, CdMnTe, InP, TIBr₂ or HGI₂ are suited to detecting x-ray quanta, since these materials have a high x-ray absorption in the energy range of medical imaging.

A layer made from scintillator material is no longer necessary. The resolution can be improved by applying a directly converting detector when the result is output in the form of annotated image data. Alternative methods for spectrally resolved CT recordings involve the operation with two tubes at different voltages (dual source), the high-frequency switching of a tube between two voltages (kV switching), the spatially selective attachment of a spectral filter either on the tube or detector side or the application of a partially transparent detector unit on another (dual layer).

In a further, advantageous embodiment of the invention, the blood loss value comprises an indication of the expected blood loss quantity and/or an indication of the expected blood loss rate and thus also an indication of the blood loss over time, which is likewise an important value for prioritizing the treatment and for introducing further emergency medical measures, which have to be carried out in minimum time due to the risk to life.

In a further advantageous embodiment of the invention, the blood loss value is integrated in a spatially resolved manner as a graphical annotation into the image data. This can be carried out in a brightness-encoded or false color-encoded manner and/or shown as textual annotation in the image data, e.g. as overlay graphics and has the advantage that the user is able to immediately identify the anatomical position and also the quantity of the blood loss as quickly and easily as possible.

In a further advantageous embodiment of the invention, the blood loss value is determined separately for each pathological accumulation of blood. This is advantageous in that not only one overall value is determined, but instead a number of individual values per bleed. It is therefore possible for a sequence to be calculated for treatments of the individual bleed sites (starting with the bleed with the highest blood loss in descending order).

In a further, advantageous embodiment of the invention, the quantification algorithm is embodied as a self-learning algorithm. It can be embodied as a multi-layer neural network. The network is trained with reference data in a training phase. For this purpose, a data storage device can be accessed, in which reference image data and reference blood loss values (e.g. as laboratory values) of other patients are stored. Furthermore, in addition or alternatively to the training, synthetically generated anatomic structures can be used, the bleeding of which is simulated with the aid of fluid-dynamic models (computational fluid dynamics). The network is then trained to identify which bleed results in which expected blood loss value.

In such cases training data, in which the blood loss value is known (or is subsequently determined from the blood image (hemoglobin content, number of blood particles)), is acquired in an input layer of the network and is calculated from the concealed layers and assigned weights in order to provide a result in the form of a blood loss value on the output layer. With the aid of this data, the neural network or the software “learns” to deduce the blood loss value from the image data. The individual layers of the network are connected to one another. After the training phase, the quantification algorithm is delivered in trained form to the customers within the scope of the method for quantifying the blood loss. The neural network thus supplies estimated values for the blood loss, which are to be expected on the basis of the training data of other patients.

One element of at least one embodiment of the present invention is therefore a training of the neural network with training data. In the execution phase, the training allows a result to be provided on the basis of the bleeding detected as pathological for the respective patient.

In a further, advantageous embodiment of the invention, the quantification algorithm comprises a segmentation of the vascular tree. It is therefore advantageous that it be possible to check more efficiently or even with another measure whether blood is outside of the vessel. The reliability of the result provided can thus be improved.

In a further, advantageous embodiment of the invention, the quantification algorithm comprises a leakage check step, which checks whether contrast agent accumulations are disposed in body regions outside of the vessels. This leakage check step is preferably implemented in the neural network. For training purposes, in particular the generated so-called iodine maps of patients without internal bleeding are used and those of patients with bleeding. The neural network then learns to differentiate between intravascular and extravascular bleeding. A similar analysis also without a preceding segmentation can be carried out using convolutional neural networks. The sought features are instead determined here by locally delimited folding operations. Other architectures of neural networks, which can likewise be used for the invention, are Deep Boltzmann Machines, autoencoders, recurrent neural networks or deep reinforcement learning.

Other approaches of machine learning, which would not be based on neural networks, e.g. support vector machines, Bayesian classifiers, k-means clustering, decision trees, convolutional neural networks, deep belief networks, deep residual learning, reinforcement learning, recurrent neural networks, inductive programming.

In a further, advantageous embodiment of the invention, the computed tomography imaging comprises two computed tomography scans in a configurable time lag, which lie in a time frame of 1 to 5 minutes. On the basis of this image data pair, the quantity of contrast agent in the bleed sites can be compared for both image data records. This provides a good estimation of the bleed speed (when indicating the measured and expected blood flow over time).

In a further, advantageous embodiment of the invention, the image analysis method comprises a material breakdown, in particular to differentiate between 2 different materials (here with and without contrast agent, in other words bleed area and other tissue). The material breakdown serves to detect all voxels, which contain the contrast agent (e.g. iodine) and to calculate what is known as an iodine map. This is possible because iodine has a specific absorption spectrum, which differs clearly from other radio-opaque substances in the image (e.g. calcium). To this end, energy-resolving detectors are preferably used in the imaging. With energy-resolving detectors, it is possible to determine an energy for each measured photon. These energy-resolving detectors divide the measured photons into two to ten energy levels. One significant advantage of these detectors is that the contrast agent breakdown is possible with the aid of each individual recording. A mask image is therefore no longer necessary. Each energy-resolved recording already intrinsically contains the information for calculating a pure contrast agent image. Furthermore, the breakdown into several materials is advantageous in that images can be generated which only indicate contrast agent, e.g. in the form of what is known as an iodine map.

According to a further embodiment, a quantification system is disclosed for quantifying blood loss on the basis of image data of a computed tomograph of contrast-agent-based computed tomography imaging of the torso of a patient. The quantification system comprises:
An image data interface for reading in the image data of the computed tomograph;
An analyser for automatically detecting intravascular (physiological) and extravascular (pathological) accumulations of blood;
A differentiator, which is determined to differentiate between pathological and physiological accumulations of blood; and
A quantifier, which is determined for calculating and outputting a blood loss value for the pathological accumulations of blood.

The blood loss value also comprises an indication of an expected blood loss. This can be output for each individual bleed.

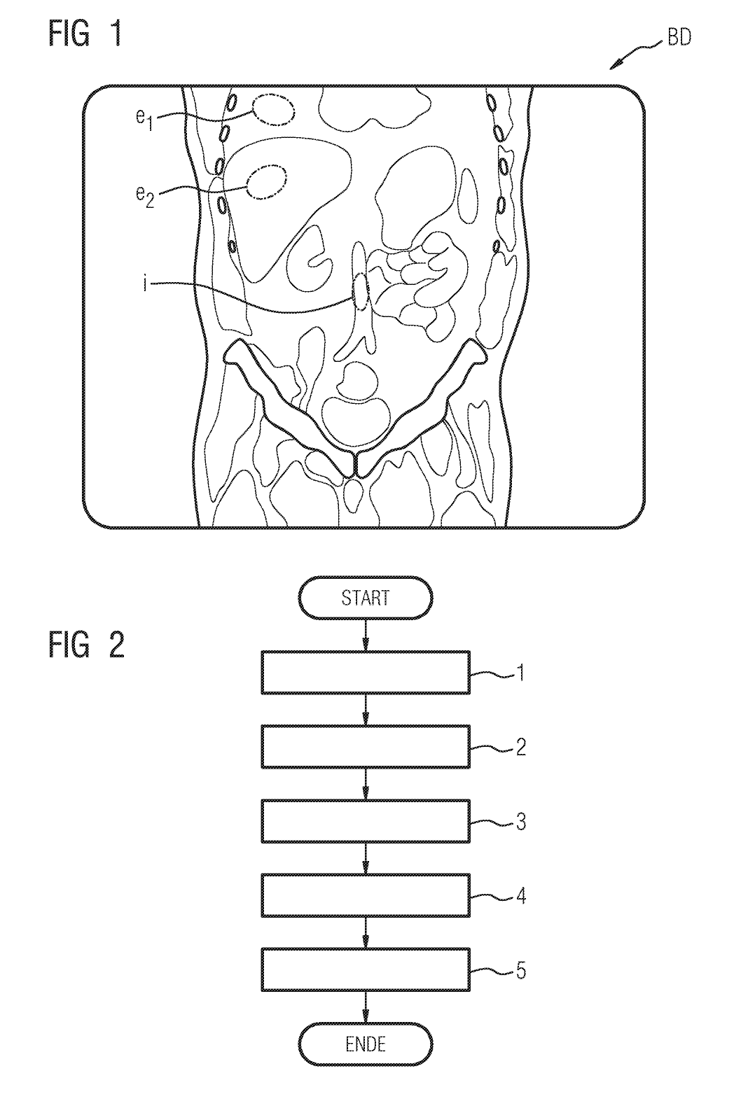

FIG. 1 shows a schematic display of an image of contrast agent-based computed tomography imaging. An abdomen or the abdominal region of a patient is shown by way of example there. It has become evident that in order to determine blood losses with assigned blood loss values, it is meaningful for the CT scan to be recorded of the torso of the patient in order to be able to acquire and evaluate all bleed sites as far as possible. The aim of the method is to generate an annotated image data record as a result, which comprises blood loss values.

In principle, blood losses, e.g. after a serious road traffic accident, can occur simultaneously at several different anatomic points in the body, which can sometimes be treated immediately (stop bleeding). The bleeding sites, which are made visible by way of a CT scan, here comprise the natural accumulations of blood in the vessels (physiological accumulations of blood), these are also referred to as intravascular accumulations of blood and pathological accumulations of blood which are also referred to as extravascular accumulations of blood. Two extravascular accumulations of blood are identified schematically in FIG. 1 with the reference characters e1, e2, which lie in the region of the right lung and in the right kidney. The heart is visible in the upper right region in FIG. 1 and the bladder is visible in the middle lower region. The aorta with the lower vena cava is shown in the central region. Since these two organs are vessels, blood in the respective organ is naturally also present in the CT image data BD so that an intravascular accumulation of blood is identified here with the reference character i.

With the quantification algorithm, a differentiation must be carried out in a preparative step to determine whether this is a pathological or physiological (natural) accumulation of blood in order to be able to usefully determine the blood loss value. This is carried out by means of a differentiation method. In such cases, in a preferred embodiment of the invention, it is possible to revert back to a 2 material breakdown, which is possible on the basis of the contrast agent-based CT imaging, because e.g. the contrast agent iodine has a specific absorption spectrum which differs significantly from other radio-opaque substances in the image (e.g. calcium). What is known as an iodine map can then be created, in which only the contrast agent accumulations of iodine are visible.

The quantification algorithm can then be carried out for the accumulations of blood detected as pathological, in order to calculate a blood loss value for a pathological accumulation of blood. The blood loss value can be specified as an overall value for the total of all lesions. It can also be output as a sequence of individual values and thus specifically output a blood loss value for each individual bleed. This is advantageous in order to prioritize the further treatment of the bleed. The quantification algorithm is preferably based on a trained neural network with a number of layers.

FIG. 2 shows a flow chart of a method for quantifying blood losses. After starting the method, the image data of a contrast-agent-assisted CT imaging is read in step 1. In such cases this may involve conventional (not energy-selective) computed tomography without distinguishing between the spectral distribution of different x-ray energies. A multi or dual-energy computed tomography can also be used however, in which two or more x-ray sources are operated with different energies and thus generate two independent tomography layer stacks. It has proven advantageous to use a directly converting detector, in particular with a directly converting semiconductor layer or an optically counting detector.

In step 2, an image analysis method is carried out, in order to acquire the pathological and physiological accumulations of blood.

Step 3 is used to determine the pathological bleeding outside of the vessels from the total quantity of bleeding acquired in step 2.

On the basis of this determination, the quantification algorithm can then be carried out in the subsequent step 4 in order to calculate the blood loss value.

In step 5 the result determined is output. Subsequently, the method can be applied repeatedly or terminated.

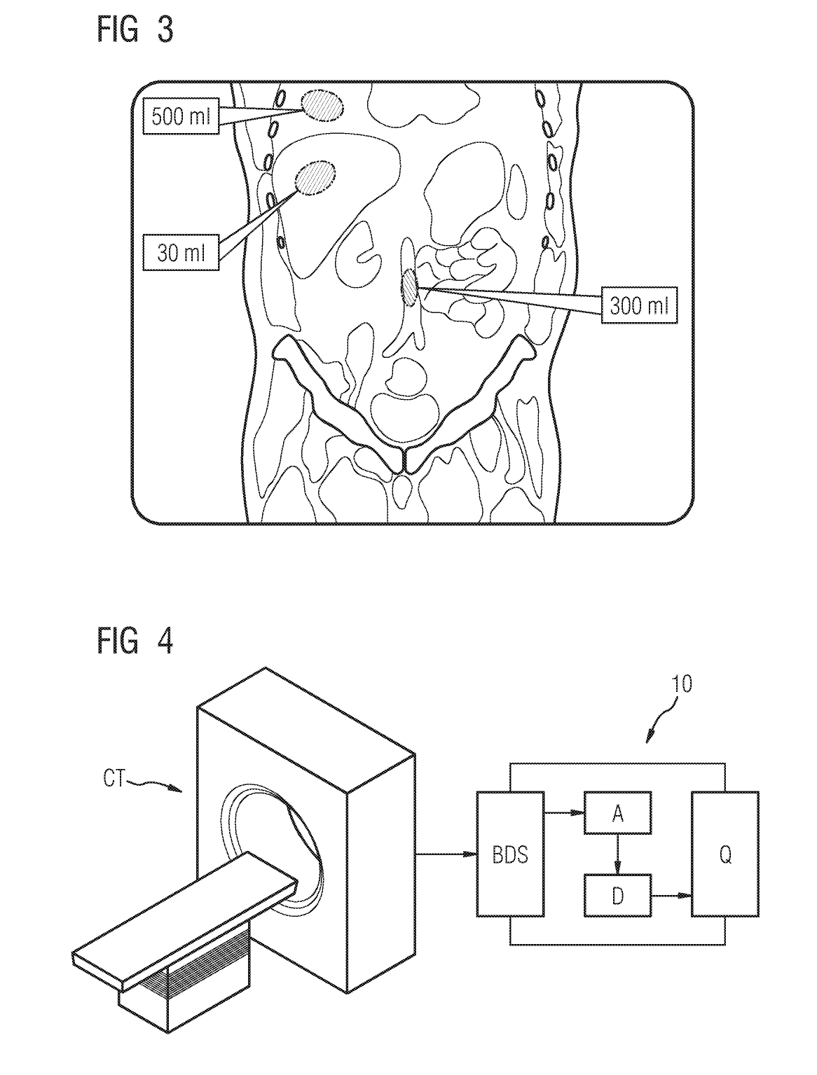

FIG. 3 shows an example of a possible kind of output for outputting step 5 of the method. The output comprises indicating the position of the anatomical bleed site, preferably directly in the image data BD, highlighted in FIG. 3 as a hatched ellipse at the respective position. The position specification or the ellipse can comprise an additional information field, which is always indicated if desired (e.g. when clicking on the bleed or moving the mouse over the corresponding point in the image) or as a presetting. The additional information field comprises the respective blood loss value of the bleeding (500, 30, 300 ml). Alternatively, the information relating to the calculated expected bleed value can also be shown visually or graphically, e.g. in false color encoding. The information in the additional information field can comprise indications of the expected quantity and/or rate of the blood loss, proposed treatment measures, risks, time aspects for the measures.

FIG. 4 shows a block diagram of a quantification system 10. The quantification system 10 interacts with a computed tomography system CT. The quantification system 10 can also be integrated directly into an imaging system or implemented on a reconstruction computer.

The quantification system 10 comprises an input interface, namely the image data interface BDS for acquiring the tomography data records BD of the computed tomograph CT. It also comprises an analyzer A for carrying out the image analysis method 2 for automatically detecting accumulations of blood and a differentiator D, which is determined to differentiate between pathological and physiological accumulations of blood and a quantifier Q which is determined for calculating and outputting a blood loss value for the pathological accumulations of blood. The quantifier therefore also serves as an output interface for the result. The result can comprise an annotated image data record, which contains the calculated blood loss value.

The principle described in the example of a polytrauma for calculating accumulations of blood in the tissue of a patient can also be applied to injuries to the tissue and other accumulations of substances in the body, such as for instance monitoring the accumulation of harmful substances or carcinogenic structures.

All features shown and explained in conjunction with individual embodiments of the invention can be provided in a different combination in the inventive subject matter in order at the same time to realize their advantageous effects.

All method steps can be implemented by apparatuses which are suited to executing the respective method step. All functions which are carried out by the objective features can be a method step of a method.

The scope of protection of the present invention is provided by the claims and is not restricted by the features explained in the description or shown in the figures.

The patent claims of the application are formulation proposals without prejudice for obtaining more extensive patent protection. The applicant reserves the right to claim even further combinations of features previously disclosed only in the description and/or drawings.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

None of the elements recited in the claims are intended to be a means-plus-function element within the meaning of 35 U.S.C. § 112(f) unless an element is expressly recited using the phrase “means for” or, in the case of a method claim, using the phrases “operation for” or “step for.”

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A method for quantifying blood loss based upon contrast agent-based computed tomography imaging of a torso of a patient, the method comprising:
reading in image data of the contrast agent-based computed tomography imaging;
applying an image analysis method to automatically detect accumulations of blood;
carrying out a differentiation method to differentiate between pathological accumulations of the blood and physiological accumulations of the blood; and
carrying out a quantification algorithm to calculate and output a blood loss value, to thereby quantify the blood loss, for the pathological accumulations of the blood.

2. The method of claim 1, wherein the contrast agent-based computed tomography imaging is achieved using a directly converting detector, including a semiconductor layer, to directly distinguish different energy levels of photons.

3. The method of claim 1, wherein the blood loss value includes an indication of at least one of expected blood loss quantity and expected blood loss rate.

4. The method of claim 1, wherein the blood loss value is integrated in a spatially-resolved manner as a graphic annotation into the image data.

5. The method of claim 1, wherein the blood loss value is determined separately for each pathological accumulation of blood.

6. The method of claim 1, wherein the quantification algorithm is embodied as a self-learning algorithm and is configured to access a data storage device, storing reference image data and reference blood loss values of other patients.

7. The method of claim 1, wherein the quantification algorithm comprises a segmentation of a vascular tree.

8. The method of claim 1, wherein the quantification algorithm comprises a leakage check step, to check whether contrast agent accumulations are disposed in body regions outside of blood vessels.

9. The method of claim 1, wherein the contrast agent-based computed tomography imaging includes two computed tomography scans in a configurable time lag.

10. The method of claim 1, wherein the image analysis method includes a 2-material breakdown for calculating an iodine map.

11. A quantification system for quantifying blood loss based upon image data of a computed tomography device of contrast agent-based computed tomography imaging of a torso of a patient, the quantification system comprising:
an image data interface to read in the image data of the computed tomography device;
an analyzer to carry out an image analysis method to automatically detect accumulations of blood;
a differentiator, determined to differentiate between pathological accumulations of the blood and physiological accumulations of the blood; and
a quantifier, determined to calculate and output a blood loss value to thereby quantify the blood loss, for pathological accumulations of the blood.

12. The method of claim 3, wherein the blood loss value includes an indication of at least one of expected blood loss quantity and expected blood loss rate.

13. The method of claim 4, wherein the blood loss value is integrated in a spatially-resolved manner as a graphic annotation into the image data.

14. The method of claim 5, wherein the blood loss value is determined separately for each pathological accumulation of blood.

15. The method of claim 2, wherein the quantification algorithm is embodied as a self-learning algorithm and is configured to access a data storage device, storing reference image data and reference blood loss values of other patients.

16. The method of claim 2, wherein the quantification algorithm comprises a segmentation of the vascular tree.

17. The method of claim 2, wherein the quantification algorithm comprises a leakage check step, to check whether contrast agent accumulations are disposed in body regions outside of the vessels.

18. The method of claim 2, wherein the contrast agent-based computed tomography imaging includes two computed tomography scans in a configurable time lag.

19. The method of claim 2, wherein the image analysis method includes a 2-material breakdown for calculating an iodine map.

20. The quantification system of claim 11, wherein the contrast agent-based computed tomography imaging is achieved using a directly converting detector, including a semiconductor layer, to directly distinguish different energy levels of photons.
